(19) United States
(12) Patent Application Publication
(10) Pub. No.: US 2013/0209534 A1
(43) Pub. Date: Aug. 15, 2013

(54) Substrate Of Polymeric Material And Method Of Carrying Out Thereof

(71) Applicants: Chiara GUALANDI, Molinella (Bologna) (IT); Maria Letizia FOCARETE, Bologna (IT); Andrea ZUCCHELLI, Crevalcore (Bologna) (IT); Maria Letizia IABICHELLA, Pisa (IT)

(72) Inventors: Chiara GUALANDI, Molinella (Bologna) (IT); Maria Letizia FOCARETE, Bologna (IT); Andrea ZUCCHELLI, Crevalcore (Bologna) (IT); Maria Letizia IABICHELLA, Pisa (IT)

(73) Assignee: MOSES S.R.L., Falciano (SM)

(21) Appl. No.: 13/487,507

(22) Filed: Jun. 4, 2012

Related U.S. Application Data
(63) Continuation of application No. PCT/SM2009/000009, filed on Nov. 3, 2009.

(51) Int. Cl. A61L 26/00

(52) U.S. Cl. CPC A61L 26/0019
USPC 424/404; 514/691; 514/456; 514/27; 514/680; 514/455; 514/468; 514/568; 514/453; 424/443; 427/2.31; 264/466

(57) ABSTRACT

A substrate of polymeric material for wound care contains at least one oleous antimicrobial and/or helping tissue regeneration substance that is selected from Hyperforin, Adhyperforin, 1-3 Diapigenin, 11-8 Diapigenin, Rutin, Quercetin, Hypericin, Azadirachtin α-β, Nimbin, Nimbidin, Salanin, Gallic Acid, Gedunin and combinations thereof.

### CROSS REFERENCE TO RELATED APPLICATIONS

This application is a continuation of PCT International Patent Application no. PCT/SM2009/000009, filed 3 Nov. 2009, the contents of which are hereby incorporated by reference.

### TECHNICAL FIELD

The invention refers in particular to a substrate of a polymeric material and to the method of carrying out thereof.

### BACKGROUND

Fibrous substrates or supports, called scaffolds, are well known materials that can be used as medical devices for tissue regeneration, where the fibres are made of natural, synthetic, biocompatible and/or biodegradable polymers, as explained for instance in the document WO2007/132186.

Scaffolds that are presently known and applied for wound healing/regeneration are characterized by several disadvantages since they are expensive (around 7-14 euros/cm² ($9-18/cm²)), they need to be stored in appropriate ambient conditions (controlled temperature and atmosphere). In addition, they must be applied in a surgical environment and their use needs a broad-spectrum antibiotic treatment.

In U.S. patent Publication no. 2003/082116, an adhesive composition is disclosed comprising a polymerizable 1,1-disubstituted ethylene monomer, particularly a-cyanoacrylate monomer, and a dual-function stabilizer which could be a herbal extract. The adhesive composition is applied to a tissue and the monomer polymerizes to form a film.

U.S. Pat. No. 6,579,543 discloses a composition having analgesic, anti-inflammatory, antioxidant and blood circulation promoting activities for application to the skin, containing St. John's wort extract.

U.S. Pat. No. 4,877,781 discloses a composition for the hemorrhoidal tissue containing gallic acid having anti-inflammatory activity.

### SUMMARY OF THE INVENTION

The main object of the present invention is to propose a substrate for wound care that is economically cheap and that can be stored also at room temperature in ambient atmosphere conditions.

Another aim of the invention is to propose a substrate that can be applied to patients in a non-sterile environment and that doesn't need a broad-spectrum antibiotic treatment.

A further aim is to propose a substrate useful for the preparation of a medicament that can be applied directly on the damaged tissue, such a medicament having better therapeutic and wound healing properties with respect to presently existing commercial products.

The polymeric material substrate is an artificial support constituted by continuous polymer fibres, preferably characterized by a diameter value lower than 1 μm.

Such a substrate contains antimicrobial and/or helping tissue regeneration substances, selected from among Hyperforin, Adhyperforin, 1-3 Diapigenin, 11-8 Diapigenin, Rutin, Quercetin, Hypericin, Azadirachtin α-β, Nimbin, Nimbidin, Salanin, Gallic Acid, Gedunin and their blends.

In a preferred embodiment of the invention, the substrate contains Hyperforin, Azadirachtin α-β, or their blends, where the mixture of Hyperforin and Azadirachtin α-β is in the form of a mixture of an extract of hypericum flowers in neem oil or hypericum oleolite mixed with neem oil. The substrate comprises a biocompatible polymeric material selected from among polymers such as: polyglycolic acid (PGA), polylactic acid (PLA), copolymers of lactic and glycolic acid (PLGA), polycaprolactone (PCL), poly(3-hydroxyalkanoate) (PHA), aliphatic polycarbonates (e.g. poly(trimethylenecarbonate) (PTMC), biodegradable polyurethanes, blends and copolymers of the above mentioned polymers, polyvinylchloride (PVC), polyethylene (PE), polypropylene (PP), polymethylmethacrylate (PMMA), polystyrene (PS),polyethyleneterephthalate (PET), polytetrafluoroethylene (PTFE), polyurethane (PU), polyamide (nylon).

The biocompatible substrate is also biodegradable or bioresorbable or bio-stable.

Further, the polymeric material is functionalized with at least one of the antimicrobial and/or helping tissue regeneration substances, in order to have one or more of such substances directly inserted into the macromolecular chain.

The polymeric material substrate is mesh-grafted.

In the preferred embodiment of the invention, the substrate is fabricated through electrospinning of the polymeric support, that later is mesh-grafted or skin-grafted.

Afterwards the polymeric material is impregnated with at least the oleous antimicrobial and/or helping tissue regeneration substance constituted preferably by a mixture of neem oil and hypericum oil.

The substrate prepared in such a way is subsequently applied onto the wound as a tissue regeneration medicament.

Another embodiment of the invention consists in the preliminary mixing of the polymeric material to be electrospun with the oleous antimicrobial and/or helping tissue regeneration substance, on the subsequent electrospinning, in the mesh-grafting or skin-grafting of the artificial substrate and following application onto the wound.

Another method of substrate preparation consists in the simultaneous electrospinning of the polymer material and the electrospraying of the oleous antimicrobial and/or helping tissue regeneration substance.

Alternatively, the artificial polymeric substrate according to the invention is fabricated through electrospinning the polymeric material which is functionalized with one or more of said oleous antimicrobial and/or helping tissue regeneration substances, in order to have one or more of such substances directly inserted in the macromolecular chain.

### BRIEF DESCRIPTION OF THE DRAWINGS

FIGS. 1a-1c show the antibacterial activity of the exemplary scaffolds using Stafilococco aureus, Pseudomonas aeruginosa and Escherichia coli bacteria incubated at a 37° C. for 1 h, 3 h and 24 h.

### DETAILED DESCRIPTION OF THE INVENTION

The invention will be further illustrated hereinafter with reference to the following example.

### Example

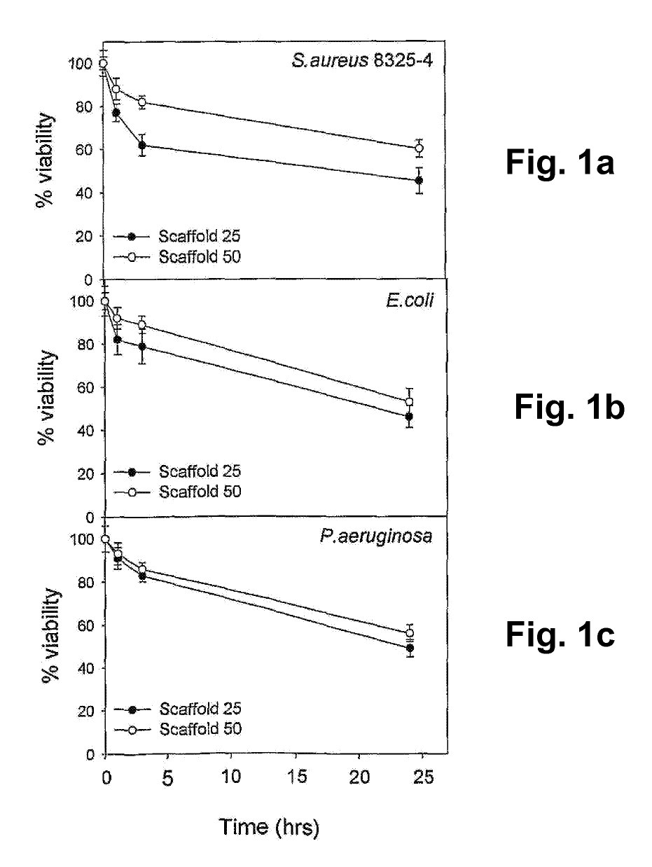

Scaffolds containing a poly(L-lactic acid) (PLLA) fibrous support and antimicrobial substances were fabricated by means of the electrospinning technology. Scaffolds characterized by two different amounts of antimicrobial substances were obtained: (i) scaffold 25 with a polymer:antimicrobial substances ratio of 75:25 wt:wt and (ii) scaffold 50 with a 50:50 wt:wt ratio. The obtained scaffolds were characterized in terms of their antibacterial activity by using Stafilococco aureus, Pseudomonas aeruginosa and Escherichia coli bacteria incubated at a 37° C. for 1 h, 3 h and 24 h, as shown in FIGS. 1a-1c.

The scaffolds show a remarkable decrease of cell viability in 24 hours (up to 60%), indicating a clear antibacterial activity. The scaffolds were mesh-grafted and directly applied on the wound.

The main advantage of the invention is to provide a cheap substrate, that also can be stored at ambient conditions and room temperature.

Another advantage is that the invention provides a substrate that can be applied to patients in non-sterile room and that doesn't need a broad-spectrum antibiotic treatment.

A further advantage is that the invention provides a substrate useful for the preparation of a medicament that can be applied directly on the damaged tissue, such medicament having better therapeutic and wound healing properties with respect to presently existing commercial products.

1. A substrate for wound care comprising:
a biocompatible and at least biodegradable or bioresorbable or bio-stable polymeric material for forming the substrate, the polymeric material selected from the group consisting of polyglycolic acid (PGA), polylactic acid (PLA), copolymers of lactic and glycolic acid (PLGA), polycaprolactone (PCL), poly(3-hydroxyalkanoate) (PHA), aliphatic polycarbonates, biodegradable polyurethanes, blends and copolymers of the above mentioned polymers, polyvinylchloride (PVC), polyethylene (PE), polypropylene (PP), polymethylmetacrylate (PMMA), polystyrene (PS), polyethylenterephthalate (PET), polytetrafluoroethylene (PTFE), polyurethane (PU), polyamide (nylon);
the substrate containing at least one oleous antimicrobial and/or helping tissue regeneration substance, said at least one substance being selected from the group consisting of Hyperforin, Adhyperforin, 1-3 Diapigenin, 11-8 Diapigenin, Rutin, Quercetin, Hypericin, Azadirachtin α-β, Nimbin, Nimbidin, Salanin, Gallic Acid, Gedunin and combinations thereof.

2. The substrate according to claim 1 wherein the at least one substance comprises Hyperforin, Azadirachtin α-β, or combinations thereof.

3. The substrate according to claim 1 wherein the at least one substance is a mixture of Hyperforin and Azadirachtin α-β in the form of a mixture of an extract of hyperforin flowers in neem oil or hypericum oleolite mixed with neem oil.

4. The substrate according to claim 1 wherein the polymeric material is functionalized with at least one of the oleous antimicrobial and/or helping tissue regeneration substances.

5. The substrate according to claim 1 wherein the biocompatible polymer material is mesh-grafted.

6. The substrate according to claim 1 wherein the polymeric material is composed of polymer fibres having a diameter of less than 1 μm.

7. A method for preparing a substrate for wound care comprising:
providing a biocompatible and at least biodegradable or bioresorbable or bio-stable polymeric material for forming the substrate, the polymeric material selected from the group consisting of polyglycolic acid (PGA), polylactic acid (PLA), copolymers of lactic and glycolic acid (PLGA), polycaprolactone (PCL), poly(3-hydroxyalkanoate) (PHA), aliphatic polycarbonates, biodegradable polyurethanes, blends and copolymers of the above mentioned polymers, polyvinylchloride (PVC), polyethylene (PE), polypropylene (PP), polymethylmetacrylate (PMMA), polystyrene (PS), polyethylenterephthalate (PET), polytetrafluoroethylene (PTFE), polyurethane (PU), polyamide (nylon);
electrospinning the polymeric material to form the substrate; and,
combining the substrate with at least one oleous antimicrobial and/or helping tissue regeneration substance, said at least one substance being selected from the group consisting of Hyperforin, Adhyperforin, 1-3 Diapigenin, 11-8 Diapigenin, Rutin, Quercetin, Hypericin, Azadirachtin α-β, Nimbin, Nimbidin, Salanin, Gallic Acid, Gedunin and combinations thereof.

8. The method according to claim 7 further comprising mesh-grafting the substrate.

9. The method of claim 7 wherein, before electrospinning to form the substrate, combining the at least one substance with the polymeric material.

10. The method of claim 7 wherein the polymer material is impregnated with the at least one substance.

11. The method of claim 7 further comprising electrospraying the at least one substance simultaneously with the electrospinning of the polymeric material.

12. A method for treating a wound comprising applying the substrate of claim 1 to a wound.

13. A method for regenerating tissue comprising applying the substrate of claim 1 to a wound requiring tissue regeneration.
